(19) United States
(12) Patent Application Publication
(10) Pub. No.: US 2023/0210964 A1
(43) Pub. Date: Jul. 6, 2023

(54) COMPOSITIONS AND METHODS FOR DETOXIFYING BACTERIAL ENDOTOXINS AND HYDROGEN SULFIDE BY RECOMBINANT FUSION ENZYMES

(71) Applicant: Sundaram Ramasamy, Mansfield, MA (US)

(72) Inventor: Sundaram Ramasamy, Mansfield, MA (US)

(21) Appl. No.: 18/008,834

(22) PCT Filed: Aug. 6, 2021
(86) PCT No.: PCT/US21/45060

§ 371 Date: Dec. 7, 2022

Related U.S. Application Data
(60) Provisional application No. 63/035,817, filed on Jun. 7, 2020.

(51) Int. Cl. A61K 38/54; C12N 9/02; C12N 9/10

(52) U.S. Cl. CPC A61K 38/54; C12N 9/0051; C12N 9/0069; C12N 9/13; C12Y 108/05; C12Y 113/11018; C12Y 208/01001

(57) ABSTRACT

Compositions comprising an engineered or non-naturally occurring polypeptides comprising two or more domains selected from Sulfide quinone oxidoreductase, Thiosulfate Sulfurtransferase and persulfide dioxygenase, and a phosphate binding motif are provided along with methods of detoxifying bacterial endotoxins and H₂S with such compositions.

### CROSS-REFERENCE TO RELATED APPLICATIONS

This application which claims the benefit of U.S. Provisional Application No. 63/035,817, filed Jun. 7, 2020. The entire contents of the above-identified applications are hereby fully incorporated herein by reference.

### REFERENCE TO AN ELECTRONIC SEQUENCE LISTING

The contents of the electronic sequence listing (“RHOG-0101WP_ST25.txt”; Size is 38,742 bytes and it was created on Aug. 5, 2021) is herein incorporated by reference in its entirety.

### TECHNICAL FIELD

The subject matter disclosed herein is generally directed to compositions and methods for detoxifying bacterial endotoxins and Hydrogen Sulfide (H₂S) by recombinant multifunctional fusion enzymes.

### BACKGROUND

Toxicity from bacterial endotoxins (e.g., LPS, LTA and LOS) and bacterial metabolite products of H₂S is a significant contributing factor to a variety of health problems. There is a need for novel reagents capable of detoxifying bacterial toxins, including endogenously and gut-bacteria-derived molecules including H₂S in vitro and in vivo.

Citation or identification of any document in this application is not an admission that such a document is available as prior art to the present invention.

### SUMMARY

In certain example embodiments, a composition is provided comprising an engineered or non-naturally occurring polypeptide comprising two or more domains selected from SQOR, TST and PDO, and a phosphate binding motif. In an embodiment, the polypeptide comprises SQOR-TST-PDO (SEQ ID NO: 9), SQOR-PDO-TST, SQOR-TST (SEQ ID NO: 7), SQOR-PDO (SEQ ID NO: 8), PDO-TST. In embodiments, TST can be replaced with a TSTD1 or a MPST domain in polypeptides disclosed herein. In embodiments, the polypeptide metabolizes sulfide, sulfite, thiosulfate, or sulfur. In an embodiment, the polypeptide metabolizes hydrogen sulfide (H₂S). In one embodiment, the polypeptide has dephosphorylation activity.

The composition of claim 1, wherein the polypeptide is capable of dephosphorylating a bacterial endotoxin. In an embodiment, the bacterial endotoxin is lipopolysaccharide (LPS), lipoteichoic acid (LTA) or lipooligosaccharide (LOS).

In an embodiment, the engineered compositions comprise a polypeptide comprising a phosphate-binding motif, which optionally comprises a sequence of CX₁X₂X₃X₄X₅R, wherein X₁ comprises 1 amino acid, X₂ comprises 1 amino acid, X₃ comprises 1 amino acid, X₄ comprises 1 amino acid, and X₅ comprises 1 amino acid. In an embodiment, the phosphate binding motif comprises one of SEQ ID NO: 12 to SEQ ID NO: 24.

One or more of the domains of the engineered or non-naturally occurring polypeptide is derived from a mammalian intestine SQOR-TST-PDO, a bovine liver SQOR-TST-PDO, a bacterial enzyme comprising one or more domains of the polypeptide, or synthetic polypeptides comprising one or more of the domains of the engineered or non-naturally occurring polypeptide. In an aspect, the compositions comprise one or a population of microorganisms producing the non-naturally occurring or engineered polypeptide. In an aspect, the microorganisms are probiotic bacteria.

The compositions and formulations disclosed herein can be a food product, a nutritional formulation, a dairy product, or a combination thereof. In one embodiment, the food product comprises a plant or a part thereof. In an embodiment, the dairy product is non-pasteurized dairy, partially pasteurized milk, a milk component, or a milk fat globule membrane component.

Pharmaceutical formulations comprising the compositions of the invention are also disclosed. The pharmaceutical formulations can further comprise a stabilizer, activator, carrier, osmotic agent, propellant, disinfectant, protective agent, diluent, nutritional agent, excipient, or a combination thereof. In an aspect, the pharmaceutical formulation is a vaccine.

Methods for treating a health condition induced by a bacterial endotoxin and H₂S are provided, the method comprising the step of administering a composition comprising an effective amount of the non-naturally occurring or engineered compositions or formulations disclosed to a subject in need thereof, wherein the non-naturally occurring or engineered polypeptide is capable of detoxifying the bacterial endotoxins and H₂S. In an aspect, the administration is performed orally, topically, or intravenously.

In one embodiment, the health condition is an LPS-H₂S-mediated, LPS-H₂S-induced, or LPS-H₂S-exacerbated disease. In an embodiment, the health condition is health condition is bowel diseases, Clostridium difficile infection, modulation of gut microbiota, alternation of bacterial over growth, Small intestinal bacterial overgrowth, antibiotic-associated diarrhea (AAD), gastrointestinal tract infections, abdominal infections, sepsis, septic shock, systemic inflammatory response syndrome, meningococcemia, trauma, hemorrhagic shock, burns, surgery, organ transplantation, liver diseases, pancreatitis, enterocolitis, periodontal diseases, pneumonia, cystic fibrosis, asthma, coronary heart diseases, congestive heart failure, kidney diseases, hypophosphatasia, hemolytic uremic syndrome, renal dialysis, preserving renal function, autoimmune diseases, rRNA import into mitochondrion malfunctions, cancers, Alzheimer's disease, aging and aging-related treatment, Radiation and radiation induced injury prevention, rheumatoid arthritis, lupus, systemic lupus erythematosus, metabolic disorders, obesity, diabetes, dyslipidemia, insulin resistant syndromes, metabolic syndrome, steatohepatitis, fatty liver, non-alcoholic fatty liver diseases, hyperglycemia, glucose intolerance, impaired glucose tolerance, insulin resistance, hyperlipidemia, hypertriglyceridemia, hypercholesterolemia, low HDL levels, high LDL levels, abdominal obesity, atherosclerosis, hypertension, and cardiovascular diseases, or a combination thereof. In an embodiment, the health condition is an infection by one or more bacteria producing the endotoxin.

Methods disclosed herein can comprise administration of compositions or formulations comprising the compositions, wherein the composition is effective to increase the number of commensal bacteria in the gastrointestinal tract, decrease the number of pathogenic bacteria in the gastrointestinal tract, or increase the number of commensal bacteria and decrease the number of pathogenic bacteria in the gastrointestinal tract, thereby modulating gastrointestinal tract flora levels in the subject.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A—H₂S metabolism by exemplary protein based thiocyanate formation demonstrates the detoxification of lipopolysaccharide-Hydrogen sulfide (LPS-H₂S) by an exemplary polypeptide according to the disclosure. The principles of the H₂S detoxification assay was based on the colorimetric method for the determination of thiocyanate formation via thiosulfate as described previously 1. As shown in FIG. 1, the exemplary fusion polypeptide rapidly metabolized H₂S to sulfite and then to thiosulfate. Metabolism of H₂S by the exemplary fusion polypeptide was tested in the presence of sodium sulfide as a donor for SQOR. As shown in FIG. 2, SQOR's final product via TST is thiosulfate. Applicant quantified thiosulfate via the formation of thiocyanate, which validated the function of both SQOR and TST (exemplary recombinant human fusion polypeptide). Briefly, the step-1 reaction mixture contains 100 mM potassium phosphate buffer, pH 7.4, 0.03% DHPC, 60 μM CoQ1, 0.1 mg ml-1 BSA, 50 mM of GSH (as acceptor) as described, 2. The reaction mixture was pre-incubated at 25° C. for 3 min, and the reaction was initiated by the simultaneous addition of 0.9 mM freshly prepared sodium sulfide as substrate and 0 to 100 units of the exemplary fusion polypeptide. After 15 min incubation, the step-1 reaction was initiated again with step-2 mixture contains 0.6 mM sulfite, 100 mM KCN, 300 mM HEPES buffer (pH 7.4) containing 150 mM of NaCl at 25° C. After 15 min incubation, reactions were terminated by the addition of 50 μl of 13.9% formaldehyde followed by 0.025 M ferric nitrate (150 μl), as described previously. The reaction product of ferric thiocyanate was detected at 460 nm in a plate reader. Control reactions were performed with the addition of heat-inactivated exemplary fusion polypeptide, and buffer control was subtracted from the values. Our preliminary result demonstrated that the exemplary fusion polypeptide is a bi-functional enzyme that uses the SQOR domain to catalyze sulfur transfer from sulfide to GSH to form GSSH and uses the modified TST domain to oxidize GSSH to thiosulfate and the exemplary fusion polypeptide has retained the independent biological activities of each enzyme. At least three independent experiments were conducted. FIG. 1B. exemplary detoxification assay.

FIG. 2—depicts the exemplary TST-1 enzyme fusion with SQOR has been re-engineered by site-directed mutagenesis to dephosphorylated LPS and the TST-2 enzyme fusion with SQOR has been re-engineered to increased metabolism of sulfite to thiosulfate. The schematic shows how an exemplary bifunctional fusion protein detoxified LPS by dephosphorylating disphosphoryl lipid A to monophosphoryl lipid A and also metabolizes bacterial toxin hydrogen sulfide (H₂S) to nontoxic sulfate (S₂O₃²⁻). Sulfide oxidation pathways adapted from Mishanina et al., 2015.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

As used herein, the term “protein” or “polypeptide” refers to a polymer of two or more of the natural amino acids or non-natural amino acids.

The term “amino acid” includes alanine (Ala or A); arginine (Arg or R); asparagines (Asn or N); aspartic acid (Asp or D); cysteine (Cys or C); glutamine (Gin or Q); glutamic acid (Glu or E); glycine (Gly or G); histidine (His or H); isoleucine (lie or I): leucine (Leu or L); lysine (Lys or K); methionine (Met or M); phenylalanine (Phe or F); proline (Pro or P); serine (Ser or S); threonine (Thr or T); tryptophan (Trp or W); tyrosine (Tyr or Y); and valine (Val or V). Non-traditional amino acids are also within the scope of the invention and include norleucine, ornithine, norvaline, homoserine, and other amino acid residue analogues such as those described in Ellman et al. Meth. Enzym. 202:301-336 (1991). To generate such non-naturally occurring amino acid residues, the procedures of Noren et al. Science 244: 182 (1989) and Ellman et al., supra, can be used. Briefly, these procedures involve chemically activating a suppressor tRNA with a non-naturally occurring amino acid residue followed by in vitro transcription and translation of the RNA. Introduction of the non-traditional amino acid can also be achieved using peptide chemistries known in the art. As used herein, the term “polar amino acid” includes amino acids that have net zero charge, but have non-zero partial charges in different portions of their side chains (e.g., M, F, W, S, Y, N, Q, C). These amino acids can participate in hydrophobic interactions and electrostatic interactions. As used herein, the term “charged amino acid” include amino acids that can have non-zero net charge on their side chains (e.g., R, K, H, E, D). These amino acids can participate in hydrophobic interactions and electrostatic interactions.

As used herein the term “linker peptide” refers to synthetic amino acid sequences that connect or link two polypeptide sequences, e.g., that link two polypeptide domains. As used herein the term “synthetic” refers to amino acid sequences that are not naturally occurring. Also naturally occurring linkers from bacterial fusion proteins are encompassed in the linker peptides.

In the context of polypeptides, a “linear sequence” or a “sequence” is the order of amino acids in a polypeptide in an amino to carboxyl terminal direction in which residues that neighbor each other in the sequence are contiguous in the primary structure of the polypeptide.

As used herein, the terms linked, fused, or fusion, are used interchangeably. These terms refer to the joining together of two more elements or components, by means including, but not limited to, chemical conjugation or recombinant means.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure, or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures, or characteristics may be combined in any suitable manner, as would be apparent to a person skilled in the art from this disclosure, in one or more embodiments. Furthermore, while some embodiments described herein include some, but not other, features included in other embodiments, combinations of features of different embodiments are meant to be within the scope of the invention. For example, in the appended claims, any of the claimed embodiments can be used in any combination.

Reference is made to [ANY RELATED PROVISIONAL, PCT, or NON-PROVISIONAL RELATED APPLICATIONS WITH RELATIVE BACKGROUND TO THE PRESENT APPLICATION]

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide compositions and methods for compositions and methods for detoxifying bacterial endotoxins such as lipopolysaccharide (LPS), Lipoteichoic acid (LTA), and Lipooligosaccharide (LOS) by dephosphorylation. In an embodiment, the compositions metabolize Hydrogen Sulfide (H₂S). In embodiments, the compositions comprise recombinant multifunctional fusion enzymes with phosphate and sulfate binding activity. An exemplary composition disclosed herein comprises an engineered or non-naturally occurring Sulfide quinone oxidoreductase-Thiosulfurtransferase (SQOR-TST) (SEQ ID NO: 7), or a functional domain thereof.

The engineered or non-naturally occurring polypeptides may comprise a sulfur-binding motif and a phosphate-binding motif The engineered or non-naturally occurring polypeptide may detoxify one or more bacterial endotoxins (e.g., LPS, LTS) by dephosphorylation as well as detoxification of H₂S. In one embodiment, the polypeptides are engineered to provide increased metabolism of sulfite to thiosulfate. Methods for removing bacterial toxins such as lipopolysaccharide, lipoteichoic acid and H₂S are provided herein. Accordingly, the present disclosure provides a protein, polynucleotide, and/or vector for use in a variety of diseases. In an embodiment, the applications include methods for treating a health condition, e.g., a condition caused by bacterial endotoxins, by administering a composition comprising the engineered or non-naturally-occurring polypeptide or a variant thereof to a subject in need thereof.

Embodiments include methods for detoxifying bacterial endotoxins in vitro, e.g., in a pharmaceutical formulation or a product generated in bacteria. Methods of removing bacterial toxins such as lipopolysaccharide, lipoteichoic acid and H₂S are provided, and can be used to reduce bacterial toxins in a biological fluid. In one embodiment, one or more of the engineered polypeptides is provided on a solid support for the capture of toxins. Coatings or layers of the one or more polypeptides comprised in or on a solid support and to cartridges, columns, and other medical apparatuses.

### Compositions

In an aspect, the present disclosure provides compositions comprising a recombinant fusion protein, also referred to herein as a non-naturally occurring or engineered polypeptide. The composition comprises a polypeptide preferably with more than one function, e.g., multifunctional. In an embodiment, the composition comprises a polypeptide with two or more domains, wherein one or more of the domains comprised in the polypeptide have been engineered to increase, enhance binding of enzymatic properties relative to a naturally occurring domain. The compositions may comprise homologs and/or orthologs of the engineered or non-naturally occurring polypeptides. The compositions may further comprise other components suitable for various applications of the exemplary polypeptides.

### Polypeptides

The polypeptides and/or domains of the polypeptide further encompass functional fragments of the domain, polypeptides, or variants thereof. The functional fragment may comprise a polypeptide or domain that includes fewer amino acid residues than the original sequence (e.g., peptide, ortholog, homolog or variant) but still confers the enzymatic activity of the original sequence of reference. In some cases, the polypeptides, domains, or variants thereof include comprise one or more polymorphisms. In embodiments, the polypeptide can comprise a sulfurtransferase domain, a phosphate binding motif, a sulfide quinone oxidoreductase (SQOR) domain, and/or a persulfide dioxygenase (PDO) domain and optionally one or more linkers. In an embodiment, the polypeptide is a TST-SQOR or SQOR-TST-PDO (SEQ ID NO: 9) or SQOR-TST (SEQ ID NO: 7) or SQOR-PDO (SEQ ID NO: 8), TST-PDO, or SQOR-PDO-TST (SEQ ID NO: 9) fusion polypeptide.

In an embodiment, the homolog and/or ortholog is a SQOR-TST (SEQ ID NO: 7) or SQOR-TST-PDO (SEQ ID NO: 9) or SQOR-TST (SEQ ID NO: 7) or SQOR-PDO (SEQ ID NO: 8), TST-PDO, SQOR-PDO-TST (SEQ ID NO: 9) polypeptide. The terms “ortholog” and “homolog” are well known in the art. By means of further guidance, a “homolog” of a protein as used herein is a protein of the same species which performs the same or a similar function as the protein it is a homologue of Homologous proteins may but need not be structurally related or are only partially structurally related. An “ortholog” of a protein as used herein is a protein of a different species which performs the same or a similar function as the protein it is an orthologue of Orthologous proteins may but need not be structurally related or are only partially structurally related. Homologs and orthologs may be identified by homology modelling (see, e.g., Greer, Science vol. 228 (1985) 1055, and Blundell et al. Eur J Biochem vol 172 (1988), 513) or “structural BLAST” (Dey F, Cliff Zhang Q, Petrey D, Honig B. Toward a “structural BLAST”: using structural relationships to infer function. Protein Sci. 2013 Apr.;22(4):359-66. doi: 10.1002/pro.2225.). Homologous proteins may but need not be structurally related or are only partially structurally related. [0036] In some embodiments, the exemplary fusion polypeptide may be a SQOR-TST (SEQ ID NO: 7) or SQOR-TST-PDO (SEQ ID NO: 9) or SQOR-TST or SQOR-PDO (SEQ ID NO: 8) or TST-PDO from a eukaryote, e.g., a mammal. Examples of mammalian SQOR-TST include mammalian intestine SQOR-TST, human SQOR-TST, or bovine SQOR-TST or SQOR-TST-PDO or SQOR-TST or SQOR-PDO or TST-PDO. In certain embodiments, the rhodanese may be a rhodanese from a prokaryote, e.g., bacteria such as E coli.

### Rhodanese/Thiosulfate Sulfurtransferase

The composition disclosed herein can comprise an engineered and/or non-naturally occurring Thiosulfate transferase (TST), also referred to herein as rhodanese. The TST or variant thereof may comprise an activity other than sulfurtransferase activity, for example, an activity comprising phosphate binding ability. In one embodiment, the TST is capable of dephosphorylating bacterial endotoxins such as lipopolysaccharide (LPS).

A rhodanese (also known as rhodanase, thiosulfate sulfurtransferase (TST), thiosulfate cyanide trans-sulfurase, and thiosulfate thiotransferase) herein refers to a mitochondrial enzyme that detoxifies cyanide (CN—), e.g., by converting it to thiocyanate (SCN—). The term rhodanese as used herein also encompasses a variant of a wildtype rhodanese, e.g., a rhodanese comprising one or more mutations (e.g., substitution, insertion, deletion and/or addition of one or more amino-acids) compared to a wildtype rhodanese counterpart. In some examples, a variant of rhodanese is an engineered, non-naturally occurring variant of rhodanese. The term rhodanese further encompasses a functional fragment of the rhodanese or a variant thereof. The functional fragment may comprise a polypeptide that includes fewer amino acid residues than the original sequence but still confers the enzymatic activity of the original sequence of reference. In some cases, rhodaneses or variants thereof include rhodaneses comprising one or more polymorphisms. Examples of rhodanese with polymorphisms include those described in Rita Cipollone et al., IUBMB Life. 2007 February;59(2):51-9; and Marouane Libiad et al., J Biol Chem. 2015 Sep. 25;290(39):23579-88, which are incorporated by reference in their entireties. An exemplary TST may be encoded by SEQ ID NO: 1 or SEQ ID NO: 4. Exemplary TST may comprise or be a functional fragment of SEQ ID NO: 3 or SEQ ID NO: 6.

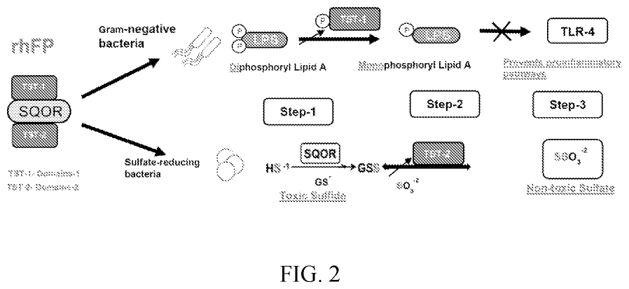

In one embodiment, the polypeptide can comprise a TST-like domain or isoform thereof, which may be a TSTD1, and MPST, or a functional fragment thereof. In one embodiment, the domain is a sulfurtransferase. Preferred embodiments comprise a TST, TSTD1, or MPST that have been engineered to comprise a phosphate binding domain. In one preferred embodiment, the phosphate binding motif is artificially synthesized within TST-1 (see FIG. 2). TST-2 (see, e.g., FIG. 2) may be mutated to work with the SQOR to accelerate or improve hydrogen sulfide metabolism. In an embodiment, the polypeptide comprises SQOR, and the SQOR is further mutated to optimize the metabolism of hydrogen sulfide along with the TST-2 mutations to provide a fusion protein comprising improved, optimized or enhances hydrogen sulfide metabolism.

In embodiments, the polypeptides can comprise Thiosulfate Sulfurtransferase Like Domain Containing 1 (TSTD1) (SEQ ID NO: 10), or TST isoform. Exemplary polypeptides may comprise SQOR-TSTD1 or TSTD1-SQOR, optionally with a linker such as (GGGS)n for example, SEQ ID NO: 25. Further exemplary polypeptides including a partial TST domain, or full length TST can be utilized where TST-1 and TST-2 are shown, see, e.g., polypeptide of FIG. 2.

In one embodiment, a modified TST like-domain containing enzyme can be used. In an embodiment, a Mercaptopyruvate sulfurtransferase (MPST) (SEQ ID NO: 11) or isoform thereof can be used in place of a TST in any of the polypeptides described herein. Therefore, SQOR-MPST or SQOR-MPST-PDO or MPST-PDO are also encompassed by the polypeptides, compositions and methods described herein.

The rhodanese or a variant thereof may have an activity other than the sulfurtransferase activity. For example, the rhodanese or an engineered or non-naturally occurring form thereof may have a dephosphorylation activity, e.g., an activity capable of dephosphorylating bacterial endotoxins such as lipopolysaccharide (LPS) or lipoteichoic acid (LTA), as well as removing phosphate group(s) from many types of molecules, including, e.g., nucleotides, CpG DNA, flagellin, proteins, ATP and ADP, and alkaloids.

The dephosphorylation activity of the TST-1 on a bacterial endotoxin may be measured by incubating the TST-1 and the endotoxin, and detecting inorganic phosphorus release, e.g., using malachite green solution by spectrophotometric absorbance readings at 650 nm wavelength), e.g., as described in Chen KT et al3., Am J Physiol Gastrointest Liver Physiol. 2010 August;299(2):G467-75, which is incorporated by reference herein in its entirety.

The rhodanese further encompasses a functional fragment of the rhodanese or a variant thereof. The functional fragment may comprise a polypeptide that includes fewer amino acid residues than the original sequence but still confers the enzymatic activity of the original sequence of reference. In some cases, rhodaneses or variants thereof include rhodaneses comprising one or more polymorphisms. Examples of rhodanese with polymorphisms include those described in Rita Cipollone et al., IUBMB Life. 2007 February;59(2):51-9; and Marouane Libiad et al., J Biol Chem. 2015 Sep. 25;290(39):23579-88, which are incorporated by reference in their entireties.

In some embodiments, the rhodanese or its variant comprises one or more phosphate-binding motifs. The phosphate-binding motifs may bind to a phosphorus atom. In some cases, the phosphate-binding motifs comprise the active-site loop of a rhodanese. In some embodiments, the phosphate-binding motifs comprise a sequence of CX₁X₂X₃X₄X₅R, wherein X₁ comprises 1 amino acid, X₂ comprises 1 amino acid, X₃ comprises 1 amino acid, X₄ comprises 1 amino acid, and X₅ comprises 1 amino acid. In some examples, X₁ is E or R. In some examples, X₂ is Y, F, E, T, K, or G. In some examples, X₃ is S or G. In some examples, X₄ is S or G. In some examples, X₅ is V or E. In some examples, X₁ is E or R; X₂ is Y, F, E, T, K, or G; X₃ is S or G; X₄ is S or G; and X₅ is V or E. Further exemplary rhodanese variants and phosphate binding motifs are described in International Patent Application PCT/US2020/035533, published as WO2020243697 at [0042]-[0047] and the sequences disclosed at SEQ ID NOS: 3-15, specifically incorporated herein by reference (corresponding to SEQ ID NOS: 12-24 in the Sequence Listing made a part of the instant application).

### Sulfide Quinone Oxidoreductase

The compositions may comprise a sulfide quinone oxidoreductase (SQOR), which may provide for H₂S detoxification, e.g., oxidation of hydrogen sulfide giving rise to thiosulfate. See, e.g., Landry, et al., ChemBioChem, 22:6 (49-960; doi:10.1002/cbic.202000661. In one preferred embodiment, the SQOR comprises a TST-2 enzyme fusion and/or TST-1 enzyme fusion with SQOR has been re-engineered to increased metabolism of sulfite to thiosulfate. Exemplary sequences of SQOR can be derived from murine sequences, e.g., NM_001162503, NM_021507.5, NR_027888.1. Exemplary sequences encoding a protein can be derived from homo sapiens, e.g., NM_021199.4, or a protein at accession NP_067022, or sequences may be derived from a bovine source, e.g., NP_001035601.2, XP_005889438.1, or XP_019824455.1. Functional fragments and variants of SQOR can be utilized in the compositions disclosed herein. In an example embodiment, the polypeptide comprises an SQOR domain in a recombinant fusion protein with either TST (or other sulfurtransferase domain, e.g. TSTD1 or MPST), PDO, or both. Exemplary polypeptides may comprise SQOR-TST (SEQ ID NO: 7), TST-1-SQOR-TST-2, SQOR-TST-PDO (SEQ ID NO: 9), SQOR-PDO-TST, SQOR-PDO (SEQ ID NO: 8), TST-SQOR, SQOR-MPST, SQOR-MPST-POD, SQOR-MPST-PDO, SQOR-PDO-MPST, MPST-SQOR, SQOR-TSTD1, SQOR-TSTD1-PDO, SQOR-PDO-TSTD1, and TSTD1-SQOR. Each of the exemplary polypeptides may comprise one or more linkers between each domain.

### Persulfide Dioxygenase

The compositions may comprise a persulfide dioxygenase (PDO), also known as ETHE1, which provide for H₂S detoxification. In one embodiment, the PDO domain is used when the application is in an oxygen rich environment. In one embodiment, the detoxification of H₂S is in bacteria, for example, in the mitochondria. See, e.g., Sattler et al., Enzymology, 290:31, 18914-18923; doi: 10.1074/jbc.M115.652537, in particular, FIG. 5 multiple sequence alignment of several PDOs and their functionally significant and conserved residues, incorporated herein specifically by reference.

In an example embodiment, the polypeptides comprise SQOR-TST-PDO (SEQ ID NO: 9), SQOR-PDO-TST (SEQ ID NO: 9), SQOR-PDO (SEQ ID NO: 8) or TST-PDO. The engineered polypeptides are capable of detoxifying lipopolysaccharide and H₂S metabolism.

### Linkers

The polypeptides of the invention may comprise at least one linker peptide. In one embodiment, a polypeptide comprises between 1 and 30 linker peptides, inclusive. In one embodiment, two or more linker peptides are present in a polypeptide of the polypeptide of the invention. In another embodiment, a polypeptide of the invention comprises 3, 4, 5, 6, 7, 8, 9 or 10 linker peptides.

Linker peptides of the invention may occur one time at a given position, or may occur multiple times (i.e., the sequence of the linker peptide may be repeated x times in sequence) at a given position in a recombinant polypeptide. For example, in one embodiment, a linker peptide of the invention is repeated between 1 and 10 times (inclusive) at a given position in a polypeptide. In another embodiment, a linker peptide occurs 2, 3, 4, 5, 6, 7, 8, 9 or 10 times at a given position in a polypeptide.

Linker peptides of the invention can be of varying lengths. In one embodiment, a linker peptide of the invention is from about 5 to about 75 amino acids in length. In another embodiment, a linker peptide of the invention is from about 5 to about 50 amino acids in length. In another embodiment, a linker peptide of the invention is from about 10 to about 40 amino acids in length. In another embodiment, a 15-linker peptide of the invention is from about 15 to about 35 amino acids in length. In another embodiment, a linker peptide of the invention is from about 15 to about 20 amino acids in length. In another embodiment, a linker peptide of the invention is from about 15 amino acids in length.

The position(s) of a linker peptide(s) of the invention may vary depending on the nature of the polypeptide to be produced. Although many specific examples of polypeptides comprising linker peptides are disclosed herein, it will be understood that linker peptides may be positioned at least wherever linker peptides are presently positioned in recombinant polypeptides. Linker peptides are so frequently used in protein engineering that they have become standard assembly parts in synthetic biology (see e.g., Anderson, J. C., et al. Journal of Biological Engineering 2010. 4: 1 and the partsregistry.org web site which lists standard biological parts used in genetic constructs).

Some examples of current, art recognized uses for linker peptides include uses in: scFv molecules (Freund et al. FEBS 1993. 320:97); single chain immunoglobulin molecules (Shun et al. 1993. PNAS. USA 90:7995); minibodies (Hun et al. 1996 Cancer Res. 56:3055); CH2 domain deleted antibodies (Mueller, B. M., et al. 1990 PNAS USA. 87:5702); single chain bispecific antibodies (Schertz et al. 2005 Cancer Res. 65:2882); full-length IgG-like bispecific antibodies (Marvin, J. S. et al. 2005 Act Pharmacology Sin 26:649 and the references cited therein as well as Michelson, J. S., et al. 2009 MAbs.1: 128 and Routt K. D. et al. 2010 Protein Eng Des Sell. 23:221); scFv fusion proteins (degree et al. 2002 British Journal of Cancer 86:811); developing protein-fragment complementation assays (Remy, I. et al. 2007 BioTechiques 42: 137), and in scFc molecules (e.g., as described in Exemplary scFc regions are disclosed in PCT Application No. PCT/US2008/006260, filed May 14, 2008, which is incorporated by reference herein).

Bioconjugation: Protein Coupling Techniques For the Biomedical Sciences Macmilan Reference, London). In one instance, a mutant can provide for enhanced binding of an FcRn binding partner for the FcRn. Also contemplated for use in the chimeric protein of the invention are peptide mimetics of at least a portion of an immunoglobulin constant region, e.g., a peptide mimetic of an Fc fragment or a peptide mimetic of an FcRn binding partner. In one embodiment, the peptide mimetic is identified using phage display or via chemical library screening (see, e.g., McCafferty et al. 1990, Nature 348:552, Kang et al. 1991, Proc. Natl. Acad. Sci. USA 88:4363; EP 0 589 877 Bl).

In another embodiment, an Fc region of the invention (e.g., an scFc region) comprises at least the portion of an Fc molecule known in the art to be required for FcyR binding.

In one embodiment, an Fc region of the invention (e.g., an scFc region) comprises at least the portion of an Fc molecule known in the art to be required for Protein A binding. In one embodiment, an Fc region of the invention (e.g., an scFc region) comprises at least the portion of an Fc molecule known in the art to be required for protein G binding. As set forth herein, it will be understood by one of ordinary skill in the art that an Fc domain may also be modified by including one or more amino acid changes (substitutions, additions or deletions) such that it varies in amino acid sequence from a wild-type Fc moiety. Many such changes or alterations are known in the art. In certain exemplary embodiments, the Fc moiety retains an effector function (e.g., FcyR binding) and in certain embodiments, the Fc moiety lacks or has reduced effector function.

Linker peptides may be attached to the N or to the C terminus (or both) of polypeptides to which they are attached.

In another embodiment, a linker peptide of the invention can be used to genetically fuse two biologically active moieties. In one embodiment, a linker peptide of the invention is used to fuse two moieties to each other, wherein neither moiety has biological activity alone, but when genetically fused, is biologically active.

The linker peptides of the instant invention traditional Gly/Ser (GS) linker peptides of the art. As used herein, the term “gly-ser linker” or “linker peptide” refers to a peptide that consists of glycine and serine residues. An exemplary gly/ser polypeptide linker comprises the amino acid sequence (Gly and Ser)n; e.g., GGGS (SEQ ID NO: 25) and GGGGS (SEQ ID NO: 27). In one embodiment, linker peptide of the instant invention comprises or consists of a Gly/Ser linker peptide with one or more amino acid substitutions, deletions, and/or additions. In example embodiments, the linker can be a Gly-Ser linker, for example as exemplified in one of SEQ ID. NO: 25-30.

In one embodiment, a polypeptide of the invention which comprises a linker peptide is a “chimeric” or “fusion” protein. Such proteins comprise a first amino acid sequence linked to a second amino acid sequence to which it is not naturally linked in nature. The amino acid sequences may normally exist in separate proteins that are brought together in the fusion polypeptide or they may normally exist in the same 30 protein but are placed in a new arrangement in the fusion polypeptide. A chimeric protein may be created, for example, by chemical synthesis, or by creating and translating a polynucleotide in which the peptide regions are encoded in the desired relationship. Exemplary chimeric polypeptides include fusion proteins comprising the linking peptides of the invention.

Polypeptides which comprise a linking peptide of the invention may be either monomeric or multimeric. For example, in one embodiment, a protein of the invention is a dimer. In one embodiment, the dimers of the invention are homodimers, comprising two identical monomeric subunits or polypeptides. In another embodiment, a dimeric polypeptide of the invention is a heterodimer, comprising two non-identical monomeric subunits or polypeptides (e.g., comprising two different biologically active moieties or one biologically active moiety only).

The skilled artisan will understand that portions of an immunoglobulin constant region for use in a polypeptide of the invention can include mutants or analogs thereof or can include chemically modified immunoglobulin constant regions (e.g., pegylated), or fragments thereof (see, e.g., Aslam and Dent 1998.

### Metabolism of Sulfur Containing Compounds

In particular embodiments, the non-naturally occurring or engineered polypeptides or variants thereof are capable of metabolic activity of H₂S, sulfite, and thiosulfate or any sulfur-containing compositions, including multiple reactive sulfur species that exist in vivo. Exemplary compositions include amino acids, DNA, and proteins. In some embodiments, the non-naturally occurring or engineered polypeptide is capable of metabolizing bacterial toxins, including gut bacteria-derived and endogenously produced by an organism sulfur containing compounds, for example, H₂S, thiosulfate, sulfur containing amino acids, and polysulfides.

### Polynucleotides and Vectors

In some embodiments, the compositions comprise one or more polynucleotides encoding the engineered or non-naturally occurring polypeptide, a functional fragment thereof, or a variant thereof. In some embodiments, the compositions may comprise a vector comprising one or more polynucleotides encoding a rhodanese, a functional fragment, or a variant thereof.

A vector may be a viral vector (e.g., adenoviral, lentiviral, or adeno-associated viral vector), where virally-derived DNA or RNA sequences are present in the vector for packaging into a virus. Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

Certain vectors may be capable of directing the expression of genes to which they are operatively-linked. Such vectors are referred to herein as “expression vectors.” Common expression vectors of utility in recombinant DNA techniques are often in the form of plasmids. A vector may be a recombinant expression vector that comprises a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. As used herein, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell).

### Formulations

In an embodiment, compositions and formulations comprise one or a population of microorganisms producing the non-naturally occurring or engineered polypeptides. In some embodiments, the microorganisms are probiotic bacteria. In some embodiments, the composition is a food product, a nutritional formulation, a dairy product, or a combination thereof. In some embodiments, the food product comprises a plant of a part thereof. In some embodiments, the dairy product is non-pasteurized dairy, partially pasteurized milk, a milk component, or a milk fat globule membrane component.

Formulations and compositions may comprise an engineered or non-naturally occurring polypeptide (e.g., recombinant fusion protein) that is deimmunized. The rhodanese may be deimmunized to render it non-immunogenic, or less immunogenic, to a given species (e.g., human). In some embodiments, the sequence of a rhodanese has been altered to eliminate one or more B- or T-cell epitopes. Deimmunization can be achieved through structural alterations to the polypeptide. Any deimmunization technique known to those skilled in the art can be employed. One suitable technique, for example, for deimmunizing proteins is described in WO 00/34317, the disclosure of which is incorporated herein in its entirety.

### Enzyme Stabilization and Pharmacokinetic Techniques

TST and/or SQOR (e.g., those isolated from natural sources as well as engineered) for use in the polypeptides of the present invention may be used in both diagnostics and disease treatment. Alternative phosphatases (e.g., polypeptides with dephosphorylation and sulfide metabolism activity) that have for example an altered (e.g., improved) specific activity, stability (e.g., in vivo T1/2, or stability in respect of storage (e.g., shelf-life)) or substrate specificity. In some examples, the rhodanese may have increased thermal stability (e.g., by altering some of the amino acids sequences (e.g., introducing mutation corresponding to E102D of human rhodanese). Stabilization of the rhodanese may be achieved through structural alterations to the polypeptide. Any stabilization technique and strategies can be used to improve rhodanese stability, including thermal stability and plasma half-life time including, but not limited to, using Fc fusion or genetic fusion to albumin that can improve pharmacokinetic technique.

### Pharmaceutical Formulations

Further provided herein include pharmaceutical formulations. The pharmaceutical formulation may comprise a non-naturally occurring or engineered polypeptide, a functional fragment thereof, or a variant thereof. In another aspect, the present disclosure provides a pharmaceutical formulation comprising the composition herein. In some embodiments, the pharmaceutical formulation further comprises a stabilizer, activator, carrier, osmotic agent, propellant, disinfectant, protective agent, diluent, nutritional agent, excipient, or a combination thereof. In some embodiments, the pharmaceutical formulation is a vaccine.

The formulations may comprise a therapeutically effective amount of the active ingredient(s) (e.g., a non-naturally occurring or engineered polypeptide, a functional fragment thereof, or a variant thereof), and a pharmaceutically acceptable carrier. The pharmaceutical formulations may also further comprise diluents, fillers, salts, buffers, stabilizers, solubilizers, activators, osmotic agents, propellants, disinfectants, protective agents, diluents, nutritional agents, excipients, and other materials well known in the art, or a combination thereof. The pharmaceutical formulation can be administered in the form of salts provided the salts are pharmaceutically acceptable. Salts may be prepared using standard procedures known to those skilled in the art of synthetic organic chemistry.

A “pharmaceutical formulation” refers to a composition that usually contains an excipient, such as a pharmaceutically acceptable carrier that is conventional in the art and that is suitable for administration to cells or to a subject. “Pharmaceutically acceptable” as used throughout this specification is consistent with the art and means compatible with the other ingredients of a pharmaceutical composition and not deleterious to the recipient thereof. In some cases, a pharmaceutical formulation is a vaccine.

The pharmaceutical formulations may comprise one or more carriers or excipients. Examples of carriers or excipients include any and all solvents, diluents, buffers (e.g., neutral buffered saline or phosphate buffered saline), solubilizers, colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners, agents for achieving a depot effect, coatings, antifungal agents, preservatives, stabilizers, antioxidants, tonicity controlling agents, absorption delaying agents, and the like. Such materials may be non-toxic and should not interfere with the activity of the cells or active components. The precise nature of the carrier or excipient or other material may depend on the route of administration. For example, the pharmaceutical formulation may be in the form of a parenterally acceptable aqueous solution, which is pyrogen-free and has suitable pH, isotonicity and stability.

The pharmaceutical formulations may be in the form of, e.g., tablets, capsules, pills, powders, granulates, suspensions, emulsions, solutions, gels including hydrogels, pastes, ointments, creams, plasters, drenches, delivery devices, injectables, implants, sprays, or aerosols.

The pharmaceutical formulations may comprise one or more pharmaceutically acceptable salts. The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. Examples of pharmaceutically acceptable salts further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. It will be understood that, as used herein, references to specific agents (e.g., neuromedin U receptor agonists or antagonists), also include the pharmaceutically acceptable salts thereof.

In some embodiments, the pharmaceutical formulations may be a sustained-release formulation. Examples of sustained-release formulations include semipermeable matrices of solid hydrophobic polymers containing the proteins of the invention, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides, copolymers of L-glutamic acid and y ethyl-L-glutamate, non-degradable ethylene-vinyl acetate, degradable lactic acid-glycolic acid copolymers, and poly-D-(−)-3-hydroxybutyric acid.

When encapsulated proteins of the invention may remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

In some embodiments, the present disclosure further comprises a delivery device or system for administering the pharmaceutical formulations to a subject. Examples of the delivery devices or systems include encapsulation in liposomes, microparticles, microcapsules, minicells, polymers, capsules, tablets, and the like. In one embodiment, delivery device is a liposome. In a liposome, the active ingredient is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. In another embodiment, the pharmaceutical formulations can be delivered in a controlled release system including, but not limited to, a delivery pump (see, for example, Saudek, et al., New Engl. J. Med. 321: 574 (1989) and a semi-permeable polymeric material (see, for example, Howard, et al., J. Neurosurg. 71: 105 (1989)). Additionally, the controlled release system can be placed in proximity of the therapeutic target (e.g., a tumor or infected tissue), thus requiring only a fraction of the systemic dose. See, for example, Goodson, In: Medical Applications of Controlled Release, 1984. (CRC Press, Boca Raton, Fla.).

In some embodiments, the pharmaceutical formulations comprise an effective amount of the active ingredient(s) (e.g., a rhodanese, a functional fragment thereof, or a variant thereof, and/or or one or more modulating agents). The term “effective amount” refers to an amount which can elicit a biological or medicinal response in a tissue, system, animal, or human that is being sought by a researcher, veterinarian, medical doctor or other clinician, and in particular can prevent or alleviate one or more of the local or systemic symptoms or features of a disease or condition being treated. In a particular embodiment, an effective amount of the active ingredient(s) may detoxify bacterial endotoxins and ameliorate a health problem caused or related to the bacterial endotoxins (e.g., their toxicity).

In some embodiments, the amount of the active ingredient(s) effective in the treatment of a particular disorder or condition depend on the nature of the disorder or condition, and may be determined by standard clinical techniques by those of skill within the art. In addition, in vitro assays may optionally be employed to help identify optimal dosage ranges. The precise dose to be employed in the formulation may also depend on the route of administration, and the overall seriousness of the disease or disorder, and may be decided according to the judgment of the practitioner and each patient's circumstances. In certain embodiments, the attending physician can administer low doses of the agent and observe the patient's response. Larger doses of the agent may be administered until the optimal therapeutic effect is obtained for the patient, and at that point the dosage is not increased further. In general, the daily dose range of a drug lie within the range known in the art for a particular drug or biologic. Effective doses may be extrapolated from dose-response curves derived from in vitro or animal model test systems. In some cases, appropriate duration of therapy using compositions of the present invention may be determined. Dosage will also vary according to the age, weight, and response of the individual patient.

In some embodiments, the composition may comprise a formulation alternative or in addition to a pharmaceutical formulation. Such alternative formulations may also comprise a rhodanese, a functional fragment thereof, or a variant thereof. Examples of such alternative formulations include food, food products, dairy products, and cells and microorganisms.

In some examples, provided herein include food or food products comprising a rhodanese, a functional fragment thereof, or a variant thereof. Examples of food or food products include meats, vegetables, plants, fruits, and flour-based products. In some cases, the food products comprise ingredients from an organism (e.g., animals or plants) that express or genetically modified to express rhodanese, a functional fragment thereof, or a variant thereof, and/or or one or more modulating agents. The food product may be from organisms (e.g., animals, plants, fungi, or microorganisms) that genetically modified to express the desired rhodanese, fragment thereof, or variants thereof. In some examples, the food product comprises an amount of SQOR-TST effective to modulate gastrointestinal tract flora levels in a subject. In some examples, the composition is a beverage product comprising an amount of rhodanese effective to modulate gastrointestinal tract flora levels in a subject.

In some examples, provided herein include dairy products, e.g., milk, butter, cheese, cream, and yogurt. In certain examples, the dairy products comprise non-pasteurized dairy or partially pasteurized milk or milk components. In a particular example, the dairy product comprises milk fat globule membrane component. In some cases, the dairy products may be from an animal (e.g., cow) that express or genetically modified to express rhodanese, a functional fragment thereof, or a variant thereof.

In some examples, provided herein include microorganisms. The microorganisms may produce (naturally or by genetic engineering), e.g., by secretion, a polypeptide as disclosed herein, e.g. SQOR-TST (SEQ ID NO: 7), a functional fragment thereof, or a variant thereof. In some embodiments, the compositions comprise probiotics. In one example, the probiotics may be in enteric coated capsules or may be genetically modified probiotic capsules secreting/producing recombinant human/bovine liver SQOR-TST or SQOR-TST-POD or SQOR-TST or SQOR-POD or TST-POD enzymes. In another example, the probiotics may be genetically modified probiotic producing recombinant human FP (rhFP-SQOR-TST or SQOR-TST-POD or SQOR-TST or SQOR-POD or TST-POD) and/or bovine liver FP. In another example, the probiotics may be genetically modified probiotic secreting/producing both human and bovine SQOR-TST enzymes. In another example, the probiotics may be genetically modified probiotic secreting/producing human SQOR-TST, or SQOR-TST-POD or SQOR-TST or SQOR-POD or TST-POD enzymes or nutritional formulation. In embodiments, the TST of any of the polypeptides can be replaced with TSTD1 or MPST, a functional portion of a TST domain or other TST-domain containing enzymes.

### Probiotics

In some embodiments, the microorganism are probiotics. By the term “probiotics” or “probiotic microorganisms” is meant non-pathogenic microorganisms that beneficially affect a subject by improving the intestinal microbial balance. Such probiotic microorganisms may preferably be anti-inflammatory microorganisms and/or from the group consisting of Lactic Acid Bacteria and Bifidobacterium spp. Lactic Acid Bacteria include all species, subspecies, and strains of the following genera: Carnobacterium, Enterococcus, Lactobacillus, Lactococcus, Leuconostoc, Oenococcus and Pediococcus. Also covered are non-pathogenic species, subspecies, and strains of the genus Streptococcus such as Streptococcus salivarius subsp. thermophilus or Streptococcus thermophilus.

The microorganisms may be of Lactobacillus spp., e.g., Lactobacillus acetotolerans, Lactobacillus acidipiscis, Lactobacillus acidophilus, Lactobacillus agilis, Lactobacillus algidus, Lactobacillus alimentarius, Lactobacillus amylolyticus, Lactobacillus amylophilus, Lactobacillus amylovorus, Lactobacillus animalis, Lactobacillus arizonensis, Lactobacillus aviarius, Lactobacillus bifermentans, Lactobacillus brevis, Lactobacillus buchneri, Lactobacillus casei, Lactobacillus coelohominis, Lactobacillus collinoides, Lactobacillus coryniformis subsp. coryniformis, Lactobacillus coryniformis subsp. torquens, Lactobacillus crispatus, Lactobacillus curvatus, Lactobacillus cypricasei, Lactobacillus delbrueckii subsp. bulgaricus, Lactobacillus delbrueckii subsp delbrueckii, Lactobacillus delbrueckii subsp. lactis, Lactobacillus durianus, Lactobacillus equi, Lactobacillus farciminis, Lactobacillus ferintoshensis, Lactobacillus fermentum, Lactobacillus formicalis, Lactobacillus fructivorans, Lactobacillus frumenti, Lactobacillus fuchuensis, Lactobacillus gallinarum, Lactobacillus gasseri, Lactobacillus graminis, Lactobacillus hamsteri, Lactobacillus helveticus, Lactobacillus helveticus subsp. jugurti, Lactobacillus heterohiochii, Lactobacillus hilgardii, Lactobacillus homohiochii, Lactobacillus intestinalis, Lactobacillus japonicus, Lactobacillus jensenii, Lactobacillus johnsonii, Lactobacillus kefiri, Lactobacillus kimchii, Lactobacillus kunkeei, Lactobacillus leichmannii, Lactobacillus letivazi, Lactobacillus lindneri, Lactobacillus malefermentans, Lactobacillus mali, Lactobacillus maltaromicus, Lactobacillus manihotivorans, Lactobacillus mindensis, Lactobacillus mucosae, Lactobacillus murinus, Lactobacillus nagelii, Lactobacillus oris, Lactobacillus panis, Lactobacillus pantheri, Lactobacillus parabuchneri, Lactobacillus paracasei subsp. paracasei, Lactobacillus paracasei subsp. pseudoplantarum, Lactobacillus paracasei subsp. tolerans, Lactobacillus parakefiri, Lactobacillus paralimentarius, Lactobacillus paraplantarum, Lactobacillus pentosus, Lactobacillus perolens, Lactobacillus plantarum, Lactobacillus pontis, Lactobacillus psittaci, Lactobacillus reuteri, Lactobacillus rhamnosus, Lactobacillus ruminis, Lactobacillus sakei, Lactobacillus salivarius, Lactobacillus salivarius subsp. salicinius, Lactobacillus salivarius subsp. salivarius, Lactobacillus sanfranciscensis, Lactobacillus sharpeae, Lactobacillus suebicus, Lactobacillus thermophilus, Lactobacillus thermotolerans, Lactobacillus vaccinostercus, Lactobacillus vaginalis, Lactobacillus versmoldensis, Lactobacillus vitulinus, Lactobacillus vermiforme, Lactobacillus zeae. In one example, the microorganism is Lactobacillus plantarum.

The microorganisms may be of Bifidobacterium spp., e.g., Bifidobacterium adolescentis, Bifidobacterium aerophilum, Bifidobacterium angulatum, Bifidobacterium animalis, Bifidobacterium asteroides, Bifidobacterium bifidum, Bifidobacterium bourn, Bifidobacterium breve, Bifidobacterium catenulatum, Bifidobacterium choerinum, Bifidobacterium coryneforme, Bifidobacterium cuniculi, Bifidobacterium dentium, Bifidobacterium gallicum, Bifidobacterium gallinarum, Bifidobacterium indicum, Bifidobacterium longum, Bifidobacterium longum bv Longum, Bifidobacterium longum bv. Infantis, Bifidobacterium longum bv. Suis, Bifidobacterium magnum, Bifidobacterium merycicum, Bifidobacterium minimum, Bifidobacterium pseudocatenulatum, Bifidobacterium pseudolongum, Bifidobacterium pseudolongum subsp. globosum, Bifidobacterium pseudolongum subsp. pseudolongum, Bifidobacterium psychroaerophilum, Bifidobacterium pullorum, Bifidobacterium ruminantium, Bifidobacterium saeculare, Bifidobacterium scardovii, Bifidobacterium subtile, Bifidobacterium thermoacidophilum, Bifidobacterium thermoacidophilum subsp. suis, Bifidobacterium thermophilum, Bifidobacterium urinalis.

In some examples, examples of the probiotics include Saccharomyces boulardii; Lactobacillus rhamnosus GG; Lactobacillus plantarum 299v; Clostridium butyricum M588; Clostridium difficile VP 20621 (non-toxigenic C. difficile strain); combination of Lactobacillus casei, Lactobacillus acidophilus; combination of Lactobacillus casei, Lactobacillus bulgaricus, Streptococcus thermophilus; combination of Lactobacillus acidophilus, Bifidobacterium bifidum; combination of Lactobacillus acidophilus, Lactobacillus bulgaricus delbrueckii subsp. bulgaricus, Lactobacillus bulgaricus casei, Lactobacillus bulgaricus plantarum, Bifidobacterium longum, Bifidobacterium infantis, Bifidobacterium breve, Streptococcus salivarius subsp. thermophilus.

### Methods of Use and Treatment

In one embodiment, methods comprise delivering a composition or formulation to a subject. Delivery or administration can be as described elsewhere herein. Methods of using the compositions and formulations to detoxify a biological fluid are also provided. In an example embodiment, one or more of the engineered polypeptides is provided on a solid support for the capture of toxins. Coatings or layers of the one or more polypeptides comprised in or on a solid support and to cartridges, columns, and other medical apparatuses. Capture technology is known in the art, and suitable solid supports and materials, including arrays, wells, platforms, columns, and beads can be utilized, and may be coated or functionalized with the polypeptide compositions of formulations provided herein for use in the capture of toxins.

In one aspect, the present disclosure provides a method for treating a health condition induced by bacterial endotoxin, the method comprising administering a composition comprising an effective amount of the non-naturally occurring or engineered polypeptide to a subject in need thereof, wherein the rhodanese is capable of detoxifying the bacterial endotoxins and H₂S. In some embodiments, the composition is the engineered, non-naturally occurring polypeptide descibed herein, or the pharmaceutical formulation comprises the engineered, non-naturally occurring polypeptide herein. In some embodiments, the composition comprises a pharmaceutical formulation. In some embodiments, the administration is performed orally, topically, or intravenously.

Such a method can comprise treatment of a mammal including a human, comprising application of a pharmaceutical composition or formulation according to the invention in a manner known for applying the polypeptide comprising pharmaceutical composition to said mammal for any of the purposes defined for the pharmaceutical composition as such or in combination. The treatment can be applied prophylactically or after trauma or suspected infection has or can have occurred. The treatment can suitably be applied before or after surgery.

In another aspect, the present disclosure provides methods for treating a health condition resulted from or related to a bacterial endotoxin. For example, the health condition may be resulted from or related to the toxicity of the bacterial endotoxin. An exemplary embodiment health condition may be LPS-H₂S-mediated, LPS-H₂S-induced, or LPS-H₂S-exacerbated diseases In some cases, the health conditions may be disorders including kidney, liver, gastrointestinal tract, abdominal infections, and lungs injuries generated by Gram-positive bacterial infection/LTA.

In general, the methods comprise administering a composition or a pharmaceutical formulation descried herein, e.g., a composition or pharmaceutical formulation comprising a rhodanese or a functional domain thereof, where the SQOR-TST (SEQ ID NO: 7) or other polypeptide described herein, which is capable of detoxifying the bacterial endotoxin, to a subject in need thereof.

In some embodiments, the health condition is an LPS-H₂S mediated, LPS-H₂S-induced, or LPS-H₂S-exacerbated disease. In some embodiments, the health condition is bowel diseases, Clostridium difficile infection, modulation of gut microbiota, alternation of bacterial over growth, Small intestinal bacterial overgrowth, antibiotic-associated diarrhea (AAD), gastrointestinal tract infections, abdominal infections, sepsis, septic shock, systemic inflammatory response syndrome, meningococcemia, trauma, hemorrhagic shock, burns, surgery, organ transplantation, liver diseases, pancreatitis, enterocolitis, periodontal diseases, pneumonia, cystic fibrosis, asthma, coronary heart diseases, congestive heart failure, kidney diseases hypophosphatasia, hemolytic uremic syndrome, renal dialysis, preserving renal function, autoimmune diseases, cancers, Alzheimer's disease, rheumatoid arthritis, lupus, systemic lupus erythematosus, metabolic disorders, obesity, diabetes, dyslipidemia, insulin resistant syndromes, metabolic syndrome, steatohepatitis, fatty liver, non-alcoholic fatty liver diseases, hyperglycemia, glucose intolerance, impaired glucose tolerance, insulin resistance, hyperlipidemia, hypertriglyceridemia, hypercholesterolemia, low HDL levels, high LDL levels, abdominal obesity, atherosclerosis, hypertension, and cardiovascular diseases, or a combination thereof. In some embodiments, the health condition is an infection by one or more bacteria producing the endotoxin. In some embodiments, the composition is effective in the treatment of viral and fungal infections. In some embodiments, the composition can be suitable for treatment of infection caused by an organism or compound of an organism, said organism being selected from the group comprising a bacterium, a fungus, a virus, or a parasite. In some embodiments, the composition is effective to increase the number of commensal bacteria in the gastrointestinal tract, decrease the number of pathogenic bacteria in the gastrointestinal tract, or increase the number of commensal bacteria and decrease the number of pathogenic bacteria in the gastrointestinal tract, thereby modulating gastrointestinal tract flora levels in the subject. In embodiments, the composition is effective to increase the number of commensal bacteria in the gastrointestinal tract, decrease the number of pathogenic bacteria in the gastrointestinal tract, or increase the number of commensal bacteria and decrease the number of pathogenic bacteria in the gastrointestinal tract, thereby modulating gut barrier function.

In some examples, the method may be used for modulating gastrointestinal tract flora levels in a subject, the method comprising administering to the subject a composition with an amount of polypeptide or variant thereof effective to increase the number of commensal bacteria in the gastrointestinal tract, decrease the number of pathogenic bacteria in the gastrointestinal tract, or increase the number of commensal bacteria and decrease the number of pathogenic bacteria in the gastrointestinal tract, thereby modulating gastrointestinal tract flora levels as well as gut wall integrity in the subject.

Specifically, as is clear from the example a pharmaceutical composition is envisaged for treating an infection caused by a bacterium. Numerous bacterial type infections can be treated such as those caused by a bacterium exhibiting multiple drug resistance (MDR). Another suitable WO 99/06440 PCT/NL97/00449 24 bacterial type infection can be treated such as that caused by a Gram-positive bacterium. Infections caused by a Gram-negative bacterium can also be treated with a pharmaceutical composition according to the invention. The invention also covers a pharmaceutical composition for treatment of infection caused by a parasite such as the parasite causing malarior Trypanosomiosis.

In another aspect, the present disclosure provides methods for making reagents, vaccines, and pharmaceutical formulations. In some embodiments, reagents, vaccines, and pharmaceutical formulations may comprise bacterial endotoxins when originally made. The methods may comprise incubating the compositions with the reagents, vaccines, and pharmaceutical formulations, wherein the composition detoxifies the bacterial endotoxins.

In certain examples, it may be desirable to have bacterial endotoxins in reagents, vaccines, or pharmaceutical formulations, but such applications may be limited by the toxicity of the endotoxins. In such cases, the compositions herein may be used to detoxify the endotoxins. The detoxified endotoxins may then be used in reagents, vaccines, or pharmaceutical formulations, e.g., as adjuvants in vaccines or vaccine antigens.

### Protein Engineering Methods

The polypeptides of the present invention comprise two or more domains which may optionally be fused directly or comprise one or more linkers between the domains. Methods of chemical conjugation (e.g., using heterobifunctional crosslinking agents) are known in the art. As used herein, the term genetically fused, genetically linked or genetic fusion refers to the co-linear, covalent linkage or attachment of two or more proteins, polypeptides, or fragments thereof via their individual peptide backbones, through genetic expression of a single polynucleotide molecule encoding those proteins, polypeptides, or fragments. Such genetic fusion results in the expression of a single contiguous genetic sequence. Preferred genetic fusions are in frame, i.e., two or more open reading frames (ORFs) are fused to form a continuous longer ORF, in a manner that maintains the correct reading frame of the original ORFs. Thus, the resulting recombinant fusion protein is a single polypeptide containing two or more protein segments that correspond to polypeptides encoded by the original ORFs (which segments are not normally so joined in nature). In this case, the single polypeptide is cleaved during processing to yield dimeric molecules comprising two polypeptide chains.

In one embodiment, a polypeptide of the invention is a binding molecule, i.e., a polypeptide that comprises a binding domain or binding site. The terms “binding domain” or “binding site”, as used herein, refer to the portion, region, or site of polypeptide that mediates specific binding with a target molecule (e.g., an antigen, ligand, receptor, substrate or inhibitor). Exemplary binding domains include an antigen binding site (e.g., a VH and/or VL domain) or molecules comprising such a binding site (e.g., an antibody), a receptor binding domain of a ligand, a ligand binding domain of a receptor or a catalytic domain. The term “ligand binding domain” as used herein refers to a native receptor (e.g., cell surface receptor) or a region or derivative thereof retaining at least a qualitative ligand binding ability, and preferably the biological activity of the corresponding native receptor. The term “receptor binding domain” as used herein refers to a native ligand or region or derivative thereof retaining at least a qualitative receptor binding ability, and preferably the biological activity of the corresponding native ligand. In one embodiment, the polypeptides of the invention have at least one binding domain specific for a molecule targeted for reduction or elimination, e.g., a cell surface antigen or a soluble antigen. In one embodiment, the binding domain comprises or consists of an antigen binding site (e.g., comprising a variable heavy chain sequence and variable light chain sequence or six CDRs from an antibody placed into alternative framework regions (e.g., human framework regions optionally comprising one or more amino acid substitutions). In one embodiment, a binding domain serves as a targeting moiety.

The polypeptides, fragments thereof, and variants thereof may be produced by recombinant techniques well known in the art. See, e.g., Green and Sambrook, Molecular Cloning: A Laboratory Manual, 4th edition (2012). These engineered polypeptides produced by recombinant technologies may be expressed from a polynucleotide. One skilled in the art will appreciate that the polynucleotides, including DNA and RNA, that encode such engineered polypeptides may be obtained from the cDNA of rhodanese and/or SQOR cDNA, fragments thereof, or variants thereof, taking into consideration the degeneracy of codon usage, and may further engineered as desired to incorporate the indicated substitutions. These polynucleotide sequences may incorporate codons facilitating transcription and translation of mRNA in microbial hosts.

Commonly used protein engineering techniques can be employed. The application of protein engineering techniques to fusion protein design has produced a number of formats that have been shown to have altered, and in some cases, improved pharmacodynamic, biodistribution, and activity profiles. Linker peptides are frequently an important part of these constructs. Linker peptides are synthetic sequences of amino acids that are commonly used to physically connect polypeptide domains. Most linker peptides are composed of repetitive modules of one or more of the amino acids glycine and serine. The standard 15 amino acid (GGGGS)₃ (SEQ ID NO: 26) or (GGGGS)n (SEQ ID NOS: 26-30) linker peptide has been well-characterized (e.g., within the context of an antibody single-chain Fv (scFv) domain) and has been shown to adopt an unstructured, flexible conformation. In addition, this linker peptide does not interfere with assembly and binding activity of the domains it connects. (Freund, C. et al., 1993. FEBS 320:97). In one embodiment, the Fc moiety is an Fc region. In one embodiment, the Fc moiety is an scFc region. In another embodiment, the polypeptide is a bispecific antibody molecule.

Host cells may be transformed with the expression or cloning vectors including different types of plasmids e.g., pCPN10/60 contains genes (cpn10 and cpn60) for cold-adapted chaperonins from Oleispira antarctica (Cpn10 and Cpn60) for protein production and cultured in conventional nutrient media modified as appropriate for inducing promoters, selecting transformants, or amplifying the genes encoding the desired sequences. The host cells used to produce the proteins of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. The media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

In some cases, the protein may be produced using cell-translation systems. For such purposes the nucleic acids encoding the polypeptide must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system.

In some cases, the protein may be produced by chemical synthesis (e.g., by the methods described in Solid Phase Peptide Synthesis, 2nd ed., 1984, The Pierce Chemical Co., Rockford, Ill.). Modifications to the protein can also be produced by chemical synthesis.

The proteins of may be purified by isolation/purification methods for proteins generally known in the field of protein chemistry. Non-limiting examples include extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, gel filtration, gel permeation chromatography, affinity chromatography, electrophoresis, countercurrent distribution, or any combinations of these. After purification, polypeptides may be exchanged into different buffers and/or concentrated by any of a variety of methods known to the art, including, but not limited to, filtration and dialysis.

The purified polypeptide is preferably at least 85% pure, more preferably at least 95% pure, and most preferably at least 98% pure. Regardless of the exact numerical value of the purity, the polypeptide is sufficiently pure for use as a pharmaceutical product.

Further embodiments are illustrated in the following Examples which are given for illustrative purposes only and are not intended to limit the scope of the invention.

### EXAMPLES

### Example 1

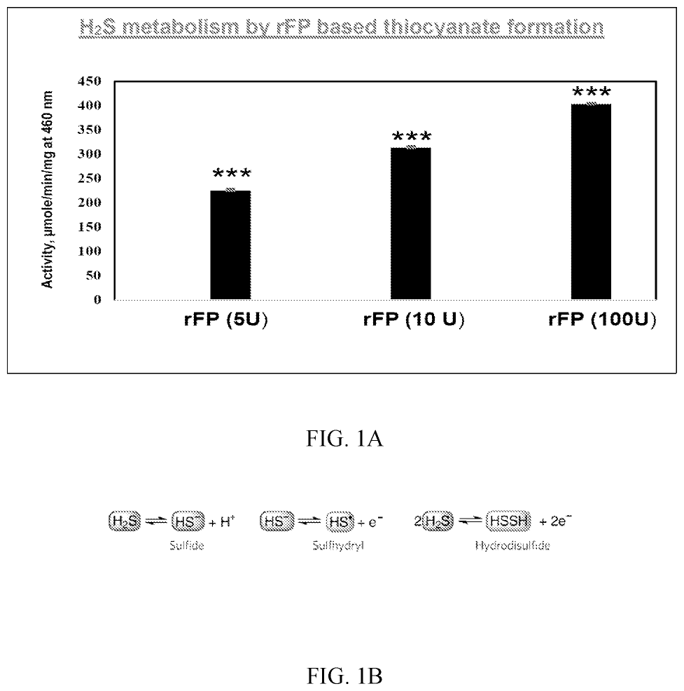

This example demonstrates that endogenous SQOR-PDO-TST, or SQOR-TST-PDO (SEQ ID NO: 9) or SQOR-TST (SEQ ID NO: 7) or SQOR-PDO (SEQ ID NO: 8) or TST-PDO detoxified LPS-H₂S encountered by the intestinal epithelium, liver, kidneys, and lungs where rhodanese expression high. First, Applicant tested the H₂S detoxification assay was based on the colorimetric method for the determination of thiocyanate formation via thiosulfate as described previously in Ramasamy et al. 2006. As shown in FIG. 1A, the exemplary fusion polypeptide rapidly metabolized H₂S to sulfite and then to thiosulfate. Metabolism of H₂S by the exemplary fusion polypeptide was tested in the presence of sodium sulfide as a donor for SQOR. As shown in FIG. 2, SQOR's final product via TST is thiosulfate. Thiosulfate was quantified via the formation of thiocyanate, which validated the function of both SQOR and TST (the exemplary fusion polypeptide). Detoxification of LPS and metabolism of bacterial toxin hydrogen sulfide to nontoxic sulfate is shown in the schematic of FIG. 2. Disphosphoryl lipid A mediates the toxic effects of LPS by activating TLR4 dependent signaling. By dephosphorylating LPS, the exemplary fusion protein blocks TLR4's downstream signaling and prevents proinflammatory pathways. As a result, the exemplary fusion protein prevents the release of inflammatory cytokines and suppresses the onset of septic symptoms. A significant reduction of LPS-H₂S levels may halt or slow the progression of sepsis.

Briefly, the step-1 reaction mixture contains 100 mM potassium phosphate buffer, pH 7.4, 0.03% DHPC, 60 μM CoQ1, 0.1 mg ml-1 BSA, 50 mM of GSH (as acceptor) as described in Libiad et al 2001. The reaction mixture was pre-incubated at 25° C. for 3 min, and the reaction was initiated by the simultaneous addition of 0.9 mM freshly prepared sodium sulfide as substrate and 0 to 100 units of rhFP. After 15 min incubation, the step-1 reaction was initiated again with step-2 mixture contains 0.6 mM sulfite, 100 mM KCN, 300 mM HEPES buffer (pH 7.4) containing 150 mM of NaCl at 25° C. After 15 min incubation, reactions were terminated by the addition of 50 μl of 13.9% formaldehyde followed by 0.025 M ferric nitrate (150 μl), as described previously Ramasamy et al 2006. The reaction product of ferric thiocyanate was detected at 460 nm in a plate reader. Control reactions were performed with the addition of heat-inactivated SQOR-TST (SEQ ID NO: 7), and buffer control was subtracted from the values. The results demonstrated that the SQOR-TST (SEQ ID NO: 7) engineered polypeptide is a bi-functional enzyme that uses the SQOR domain to catalyze sulfur transfer from sulfide to GSH to form GSSH and uses the modified TST domain to oxidize GSSH to thiosulfate and SQOR-TST (SEQ ID NO: 7) has retained the independent biological activities of each enzyme.

### References

Ramasamy S, Singh S, Taniere P, Langman M. J., Eggo M. C. Sulfide-detoxifying enzymes in the human colon are decreased in cancer and upregulated in differentiation. Am J Physiol Gastrointest Liver Physiol. 2006;291(2):G288-296.

Libiad M, Yadav P. K., Vitvitsky V, Martinov M, Banerjee R. Organization of the human mitochondrial hydrogen sulfide oxidation pathway. J Biol Chem. 2014;289(45):30901-30910.

Chen K. T., Malo M. S., Moss A. K., et al. Identification of specific targets for the gut mucosal defense factor intestinal alkaline phosphatase. Am J Physiol Gastrointest Liver Physiol. 2010;299(2):G467-475.

### ***

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A composition comprising an engineered or non-naturally occurring polypeptide comprising two or more domains selected from: sulfide quinone oxidoreductase (SQOR), Thiosulfate sulfurtransferase (TST) or sulfurtransferase domain engineered to comprise a phosphate binding motif, and persulfide dioxygenase (PDO.

2. The composition of claim 1, wherein the polypeptide comprises SQOR-TST-PDO, SQOR-PDO-TST, SQOR-TST, SQOR-PDO, PDO-TST, SEQ ID NO: 7, SEQ ID NO: 8, or SEQ ID NO: 9.

3. The composition of claim 1, wherein the sulfurtransferase domain comprises TSTD1, MPST or a variant or functional fragment thereof.

4. (canceled)

5. The composition of claim 1, wherein the polypeptide metabolizes sulfide, sulfite, thiosulfate, or sulfur, and optionally wherein the polypeptide has dephosphorylation activity, or the polypeptide metabolizes hydrogen sulfide (H₂S).

6. (canceled)

7. (canceled)

8. The composition of claim 1, wherein the polypeptide is capable of dephosphorylating a bacterial endotoxin, and optionally wherein the polypeptide is capable of metabolism of hydrogen sulfide, or the bacterial endotoxin is lipopolysaccharide (LPS), lipoteichoic acid (LTA) or lipooligosaccharide (LOS).

9. (canceled)

10. (canceled)

11. The composition of claim 1, wherein the phosphate-binding motif comprises a sequence of CX₁X₂X₃X₄X₅R, wherein X₁ comprises 1 amino acid, X₂ comprises 1 amino acid, X₃ comprises 1 amino acid, X₄ comprises 1 amino acid, and X₅ comprises 1 amino acid, and optionally wherein the phosphate binding motif is comprised within a TST domain of the polypeptide, preferably wherein the TST domain comprises SEQ ID NO: 3.

12. (canceled)

13. The composition of claim 1, wherein one or more of the domains of the engineered or non-naturally occurring polypeptide is derived from a mammalian intestine SQOR-TST-PDO, a bovine liver SQOR-TST-PDO, a bacterial enzyme comprising one or more domains of the polypeptide, synthetic polypeptides comprising one or more of the domains of the engineered or non-naturally occurring polypeptide, or engineered recombinant human polypeptide derived from a cell line or microorganism.

14. The composition of claim 1, comprising one or a population of microorganisms producing the non-naturally occurring or engineered polypeptide.

15. The composition of claim 14, wherein the microorganisms are probiotic bacteria.

16. The composition of claim 1, wherein the composition is a food product, a nutritional formulation, a dairy product, or a combination thereof.

17. The composition of claim 16, wherein the food product comprises a plant of a part thereof, or wherein the dairy product is non-pasteurized dairy, partially pasteurized milk, a milk component, or a milk fat globule membrane component.

18. (canceled)

19. A pharmaceutical formulation comprising the composition of claim 1.

20. The pharmaceutical formulation of claim 19, further comprising a stabilizer, activator, carrier, osmotic agent, propellant, disinfectant, protective agent, diluent, nutritional agent, excipient, or a combination thereof, or wherein the pharmaceutical formulation is a vaccine.

21. (canceled)

22. A method for treating a health condition induced by a bacterial endotoxin and H₂S, the method comprising administering a composition comprising an effective amount of the non-naturally occurring or engineered composition of claim 1, to a subject in need thereof, wherein the non-naturally occurring or engineered polypeptide is capable of detoxifying the bacterial endotoxins and H₂S.

23. The method of claim 22, wherein the administration is performed orally, topically, or intravenously.

24. The method of claim 22, wherein the health condition is:
an LPS-H₂S-mediated, LPS-H₂S-induced, or LPS-H₂S-exacerbated disease;
a bowel disease, a Clostridium difficile infection, a modulation of gut microbiota, an alternation of bacterial over growth, a small intestinal bacterial overgrowth, antibiotic-associated diarrhea (AAD), a gastrointestinal tract infection, an abdominal infection, sepsis, septic shock, systemic inflammatory response syndrome, meningococcemia, trauma, hemorrhagic shock, a burn, a surgery, an organ transplantation, a liver disease, pancreatitis, enterocolitis, a periodontal disease, pneumonia, cystic fibrosis, asthma, coronary heart diseases, congestive heart failure, a kidney disease, hypophosphatasia, hemolytic uremic syndrome, renal dialysis, preserving renal function, an autoimmune disease, a rRNA import into mitochondrion malfunction, a cancer, Alzheimer's disease, an aging and aging-related treatment, radiation and radiation induced injury prevention, rheumatoid arthritis, lupus, systemic lupus erythematosus, a metabolic disorder, obesity, diabetes, dyslipidemia, an insulin resistant syndrome, metabolic syndrome, steatohepatitis, fatty liver, a non-alcoholic fatty liver disease, hyperglycemia, glucose intolerance, impaired glucose tolerance, insulin resistance, hyperlipidemia, hypertriglyceridemia, hypercholesterolemia, a low HDL level, a high LDL level, abdominal obesity, atherosclerosis, hypertension, or a cardiovascular disease, or a combination thereof; or
an infection by one or more bacteria producing the endotoxin.

25. (canceled)

26. (canceled)

27. The method of claim 22, wherein the composition is effective to increase the number of commensal bacteria in the gastrointestinal tract, decrease the number of pathogenic bacteria in the gastrointestinal tract, or increase the number of commensal bacteria and decrease the number of pathogenic bacteria in the gastrointestinal tract, thereby modulating gastrointestinal tract flora levels in the subject and/or modulating gut barrier function.

28. A method for treating a health condition induced by a bacterial endotoxin and H₂S, the method comprising administering a composition comprising an effective amount of the formulation of claim 19, to a subject in need thereof, wherein the non-naturally occurring or engineered polypeptide is capable of detoxifying the bacterial endotoxins and H₂S.

29. The method of claim 28, wherein the health condition is:
an LPS-H₂S-mediated, LPS-H₂S-induced, or LPS-H₂S-exacerbated disease;
a bowel disease, a Clostridium difficile infection, a modulation of gut microbiota, an alternation of bacterial over growth, a small intestinal bacterial overgrowth, antibiotic-associated diarrhea (AAD), a gastrointestinal tract infection, an abdominal infection, sepsis, septic shock, systemic inflammatory response syndrome, meningococcemia, trauma, hemorrhagic shock, a burn, a surgery, an organ transplantation, a liver disease, pancreatitis, enterocolitis, a periodontal disease, pneumonia, cystic fibrosis, asthma, coronary heart diseases, congestive heart failure, a kidney disease, hypophosphatasia, hemolytic uremic syndrome, renal dialysis, preserving renal function, an autoimmune disease, a rRNA import into mitochondrion malfunction, a cancer, Alzheimer's disease, an aging and aging-related treatment, radiation and radiation induced injury prevention, rheumatoid arthritis, lupus, systemic lupus erythematosus, a metabolic disorder, obesity, diabetes, dyslipidemia, an insulin resistant syndrome, metabolic syndrome, steatohepatitis, fatty liver, a non-alcoholic fatty liver disease, hyperglycemia, glucose intolerance, impaired glucose tolerance, insulin resistance, hyperlipidemia, hypertriglyceridemia, hypercholesterolemia, a low HDL level, a high LDL level, abdominal obesity, atherosclerosis, hypertension, or a cardiovascular disease, or a combination thereof; or
an infection by one or more bacteria producing the endotoxin.

30. The method of claim 28, wherein the composition is effective to increase the number of commensal bacteria in the gastrointestinal tract, decrease the number of pathogenic bacteria in the gastrointestinal tract, or increase the number of commensal bacteria and decrease the number of pathogenic bacteria in the gastrointestinal tract, thereby modulating gastrointestinal tract flora levels in the subject and/or modulating gut barrier function.


SEQUENCE LISTING (30 sequences)

SEQ ID NO: 1 - DNA, 897 bp, Artificial Sequence
  Synthetic polynucleotide
atggttcatcaggttctgtatcgtgcactggttagcaccaaatggctggcagaaagcattcgtaccggtaaattaggtccgggtctgcgtgttctggatgcaagctggtatagtccgggtacacgtgaagcacgtaaagaatatctggaacgtcatgttccgggtgcaagctttttcgatattgaagaatgtcgtgataccgccagtccgtatgaaatgatgctgccgagcgaagcaggttttgcagaatatgttggtcgtctgggtattagcaatcatacccatgttgttgtgtatgatggtgaacatctgggtagcttttatgcaccgcgtgtttggtggatgtttcgtgtttttggtcatcgtaccgttagcgttctgaatggtggttttcgtaattggctgaaagaaggtcatccggttaccagcgaaccgagccgtccggaaccggcagtttttaaagcaaccctggatcgtagcctgctgaaaacctatgaacaggttctggaaaatctggaaagcaaacgttttcagctggttgatagccgtagccagggtcgttttctgggcaccgaacctgaaccggatgcagttggtctggatagcggtcatattcgtggtgcagttaatatgccgtttatggattttctgaccgaagatggctttgaaaaaggtccggaagaactgcgtgcactgtttcagaccaagaaagttgatctgagccagccgctgattgcaacctgtgaattttcaagcgaacgtgcatgtcatgttgcactggcagcatatctgtgtggcaaaccggatgttgcagtttatgatggtagctggtcagaatggtttcgtcgtgcaccgcctgaaagccgtgtgagccagggtaaaagcgaaaaagcataa

SEQ ID NO: 2 - DNA, 897 bp, Artificial Sequence
  Synthetic polynucleotide complementary to SEQ ID NO 1
ttatgctttttcgcttttaccctggctcacacggctttcaggcggtgcacgacgaaaccattctgaccagctaccatcataaactgcaacatccggtttgccacacagatatgctgccagtgcaacatgacatgcacgttcgcttgaaaattcacaggttgcaatcagcggctggctcagatcaactttcttggtctgaaacagtgcacgcagttcttccggacctttttcaaagccatcttcggtcagaaaatccataaacggcatattaactgcaccacgaatatgaccgctatccagaccaactgcatccggttcaggttcggtgcccagaaaacgaccctggctacggctatcaaccagctgaaaacgtttgctttccagattttccagaacctgttcataggttttcagcaggctacgatccagggttgctttaaaaactgccggttccggacggctcggttcgctggtaaccggatgaccttctttcagccaattacgaaaaccaccattcagaacgctaacggtacgatgaccaaaaacacgaaacatccaccaaacacgcggtgcataaaagctacccagatgttcaccatcatacacaacaacatgggtatgattgctaatacccagacgaccaacatattctgcaaaacctgcttcgctcggcagcatcatttcatacggactggcggtatcacgacattcttcaatatcgaaaaagcttgcacccggaacatgacgttccagatattctttacgtgcttcacgtgtacccggactataccagcttgcatccagaacacgcagacccggacctaatttaccggtacgaatgctttctgccagccatttggtgctaaccagtgcacgatacagaacctgatgaaccat

SEQ ID NO: 3 - protein, 298 aa, Artificial Sequence
  Synthetic polypeptide
MVHQVLYRALVSTKWLAESIRTGKLGPGLRVLDASWYSPGTREARKEYLERHVPGASFFDIEECRDTASPYEMMLPSEAGFAEYVGRLGISNHTHVVVYDGEHLGSFYAPRVWWMFRVFGHRTVSVLNGGFRNWLKEGHPVTSEPSRPEPAVFKATLDRSLLKTYEQVLENLESKRFQLVDSRSQGRFLGTEPEPDAVGLDSGHIRGAVNMPFMDFLTEDGFEKGPEELRALFQTKKVDLSQPLIATCEFSSERACHVALAAYLCGKPDVAVYDGSWSEWFRRAPPESRVSQGKSEKA

SEQ ID NO: 4 - DNA, 894 bp, Artificial Sequence
  Synthetic polynucleotide
atggttcatcaggttctgtatcgtgcactggttagcaccaaatggctggcagaaagcattcgtaccggtaaattaggtccgggtctgcgtgttctggatgcaagctggtatagtccgggtacacgtgaagcacgtaaagaatatctggaacgtcatgttccgggtgcaagctttttcgatattgaagaatgtcgtgataccgccagtccgtatgaaatgatgctgccgagcgaagcaggttttgcagaatatgttggtcgtctgggtattagcaatcatacccatgttgttgtgtatgatggtgaacatctgggtagcttttatgcaccgcgtgtttggtggatgtttcgtgtttttggtcatcgtaccgttagcgttctgaatggtggttttcgtaattggctgaaagaaggtcatccggttaccagcgaaccgagccgtccggaaccggcagtttttaaagcaaccctggatcgtagcctgctgaaaacctatgaacaggttctggaaaatctggaaagcaaacgttttcagctggttgatagccgtagccagggtcgttttctgggcaccgaacctgaaccggatgcagttggtctggatagcggtcatattcgtggtgcagttaatatgccgtttatggattttctgaccgaagatggctttgaaaaaggtccggaagaactgcgtgcactgtttcagaccaagaaagttgatctgagccagccgctgattgcaacctgtcgtaaaggtgttaccgcatgtcatgttgcactggcagcatatctgtgtggcaaaccggatgttgcagtttatgatggtagctggtcagaatggtttcgtcgtgcaccgcctgaaagccgtgtgagccagggtaaaagcgaaaaagcataa

SEQ ID NO: 5 - DNA, 894 bp, Artificial Sequence
  Synthetic polynucleotide complementary to SEQ ID NO 4
ttatgctttttcgcttttaccctggctcacacggctttcaggcggtgcacgacgaaaccattctgaccagctaccatcataaactgcaacatccggtttgccacacagatatgctgccagtgcaacatgacatgcggtaacacctttacgacaggttgcaatcagcggctggctcagatcaactttcttggtctgaaacagtgcacgcagttcttccggacctttttcaaagccatcttcggtcagaaaatccataaacggcatattaactgcaccacgaatatgaccgctatccagaccaactgcatccggttcaggttcggtgcccagaaaacgaccctggctacggctatcaaccagctgaaaacgtttgctttccagattttccagaacctgttcataggttttcagcaggctacgatccagggttgctttaaaaactgccggttccggacggctcggttcgctggtaaccggatgaccttctttcagccaattacgaaaaccaccattcagaacgctaacggtacgatgaccaaaaacacgaaacatccaccaaacacgcggtgcataaaagctacccagatgttcaccatcatacacaacaacatgggtatgattgctaatacccagacgaccaacatattctgcaaaacctgcttcgctcggcagcatcatttcatacggactggcggtatcacgacattcttcaatatcgaaaaagcttgcacccggaacatgacgttccagatattctttacgtgcttcacgtgtacccggactataccagcttgcatccagaacacgcagacccggacctaatttaccggtacgaatgctttctgccagccatttggtgctaaccagtgcacgatacagaacctgatgaaccat

SEQ ID NO: 6 - protein, 297 aa, Homo sapiens
MVHQVLYRALVSTKWLAESIRTGKLGPGLRVLDASWYSPGTREARKEYLERHVPGASFFDIEECRDTASPYEMMLPSEAGFAEYVGRLGISNHTHVVVYDGEHLGSFYAPRVWWMFRVFGHRTVSVLNGGFRNWLKEGHPVTSEPSRPEPAVFKATLDRSLLKTYEQVLENLESKRFQLVDSRSQGRFLGTEPEPDAVGLDSGHIRGAVNMPFMDFLTEDGFEKGPEELRALFQTKKVDLSQPLIATCRKGVTACHVALAAYLCGKPDVAVYDGSWSEWFRRAPPESRVSQGKSEKA

SEQ ID NO: 7 - protein, 708 aa, Artificial Sequence
  Fusion protein (SQOR-linker-TST)
  MISC_FEATURE (410)..(411): Gly-Ser Linker or any other linker of any length
MNHYEVLVLGGGSGGITMAARMKRKVGAENVAIVEPSERHFYQPIWTLVGAGAKQLSSSGRPTASVIPSGVEWIKARVTELNPDKNCIHTDDDEKISYRYLIIALGIQLDYEKIKGLPEGFAHPKIGSNYSVKTVEKTWKALQDFKEGNAIFTFPNTPVKCAGAPQKIMYLSEAYFRKTGKRSKANIIFNTSLGAIFGVKKYADALQEIIQERNLTVNYKKNLIEVRADKQEAVFENLDKPGETQVISYEMLHVTPPMSPPDVLKTSPVADAAGWVDVDKETLQHRRYPNVFGIGDCTNLPTSKTAAAVAAQSGILDRTISVIMKNQTPTKKYDGYTSCPLVTGYNRVILAEFDYKAEPLETFPFDQSKERLSMYLMKADLMPFLYWNMMLRGYWGGPAFLRKLFHLGMSMVHQVLYRALVSTKWLAESIRTGKLGPGLRVLDASWYSPGTREARKEYLERHVPGASFFDIEECRDTASPYEMMLPSEAGFAEYVGRLGISNHTHVVVYDGEHLGSFYAPRVWWMFRVFGHRTVSVLNGGFRNWLKEGHPVTSEPSRPEPAVFKATLDRSLLKTYEQVLENLESKRFQLVDSRSQGRFLGTEPEPDAVGLDSGHIRGAVNMPFMDFLTEDGFEKGPEELRALFQTKKVDLSQPLIATCEFSSERACHVALAAYLCGKPDVAVYDGSWSEWFRRAPPESRVSQGKSEKA

SEQ ID NO: 8 - protein, 664 aa, Artificial Sequence
  Fusion protein (SQOR-linker-ETHE1 or its isoforms)
  MISC_FEATURE (410)..(411): Gly-Ser Linker or any other linker of any length
MNHYEVLVLGGGSGGITMAARMKRKVGAENVAIVEPSERHFYQPIWTLVGAGAKQLSSSGRPTASVIPSGVEWIKARVTELNPDKNCIHTDDDEKISYRYLIIALGIQLDYEKIKGLPEGFAHPKIGSNYSVKTVEKTWKALQDFKEGNAIFTFPNTPVKCAGAPQKIMYLSEAYFRKTGKRSKANIIFNTSLGAIFGVKKYADALQEIIQERNLTVNYKKNLIEVRADKQEAVFENLDKPGETQVISYEMLHVTPPMSPPDVLKTSPVADAAGWVDVDKETLQHRRYPNVFGIGDCTNLPTSKTAAAVAAQSGILDRTISVIMKNQTPTKKYDGYTSCPLVTGYNRVILAEFDYKAEPLETFPFDQSKERLSMYLMKADLMPFLYWNMMLRGYWGGPAFLRKLFHLGMSMAEAVLRVARRQLSQRGGSGAPILLRQMFEPVSCTFTYLLGDRESREAVLIDPVLETAPRDAQLIKELGLRLLYAVNTHCHADHITGSGLLRSLLPGCQSVISRLSGAQADLHIEDGDSIRFGRFALETRASPGHTPGCVTFVLNDHSMAFTGDALLIRGCGRTDFQQGCAKTLYHSVHEKIFTLPGDCLIYPAHDYHGFTVSTVEEERTLNPRLTLSCEEFVKIMGNLNLPKPQQIDFAVPANMRCGVQTPTA

SEQ ID NO: 9 - protein, 962 aa, Artificial Sequence
  Fusion protein (SQOR-linker-ETHE1-linker-TST)
  MISC_FEATURE (410)..(411): Gly-Ser Linker or any other linker of any length
  MISC_FEATURE (664)..(665): Gly-Ser Linker or any other linker of any length
MNHYEVLVLGGGSGGITMAARMKRKVGAENVAIVEPSERHFYQPIWTLVGAGAKQLSSSGRPTASVIPSGVEWIKARVTELNPDKNCIHTDDDEKISYRYLIIALGIQLDYEKIKGLPEGFAHPKIGSNYSVKTVEKTWKALQDFKEGNAIFTFPNTPVKCAGAPQKIMYLSEAYFRKTGKRSKANIIFNTSLGAIFGVKKYADALQEIIQERNLTVNYKKNLIEVRADKQEAVFENLDKPGETQVISYEMLHVTPPMSPPDVLKTSPVADAAGWVDVDKETLQHRRYPNVFGIGDCTNLPTSKTAAAVAAQSGILDRTISVIMKNQTPTKKYDGYTSCPLVTGYNRVILAEFDYKAEPLETFPFDQSKERLSMYLMKADLMPFLYWNMMLRGYWGGPAFLRKLFHLGMSMAEAVLRVARRQLSQRGGSGAPILLRQMFEPVSCTFTYLLGDRESREAVLIDPVLETAPRDAQLIKELGLRLLYAVNTHCHADHITGSGLLRSLLPGCQSVISRLSGAQADLHIEDGDSIRFGRFALETRASPGHTPGCVTFVLNDHSMAFTGDALLIRGCGRTDFQQGCAKTLYHSVHEKIFTLPGDCLIYPAHDYHGFTVSTVEEERTLNPRLTLSCEEFVKIMGNLNLPKPQQIDFAVPANMRCGVQTPTAMVHQVLYRALVSTKWLAESIRTGKLGPGLRVLDASWYSPGTREARKEYLERHVPGASFFDIEECRDTASPYEMMLPSEAGFAEYVGRLGISNHTHVVVYDGEHLGSFYAPRVWWMFRVFGHRTVSVLNGGFRNWLKEGHPVTSEPSRPEPAVFKATLDRSLLKTYEQVLENLESKRFQLVDSRSQGRFLGTEPEPDAVGLDSGHIRGAVNMPFMDFLTEDGFEKGPEELRALFQTKKVDLSQPLIATCEFSSERACHVALAAYLCGKPDVAVYDGSWSEWFRRAPPESRVSQGKSEKA

SEQ ID NO: 10 - protein, 115 aa, Homo sapiens
MAGAPTVSLPELRSLLASGRARLFDVRSREEAAAGTIPGALNIPVSELESALQMEPAAFQALYSAEKPKLEDEHLVFFCQMGKRGLQATQLARSLGYTGARNYAGAYREWLEKES

SEQ ID NO: 11 - protein, 297 aa, Homo sapiens
MASPQLCRALVSAQWVAEALRAPRAGQPLQLLDASWYLPKLGRDARREFEERHIPGAAFFDIDQCSDRTSPYDHMLPGAEHFAEYAGRLGVGAATHVVIYDASDQGLYSAPRVWWMFRAFGHHAVSLLDGGLRHWLRQNLPLSSGKSQPAPAEFRAQLDPAFIKTYEDIKENLESRRFQVVDSRATGRFRGTEPEPRDGIEPGHIPGTVNIPFTDFLSQEGLEKSPEEIRHLFQEKKVDLSKPLVATCGSGVTACHVALGAYLCGKPDVPIYDGSWVEWYMRARPEDVISEGRGKTH

SEQ ID NO: 12 - protein, 7 aa, Artificial Sequence
  Synthetic
CEYSGVR

SEQ ID NO: 13 - protein, 7 aa, Artificial Sequence
  Synthetic
CEFSGVR

SEQ ID NO: 14 - protein, 7 aa, Artificial Sequence
  Synthetic
CREGSVR

SEQ ID NO: 15 - protein, 7 aa, Artificial Sequence
  Synthetic
CRYSGVR

SEQ ID NO: 16 - protein, 7 aa, Artificial Sequence
  Synthetic
CETSGVR

SEQ ID NO: 17 - protein, 7 aa, Artificial Sequence
  Synthetic
CEYSGVR

SEQ ID NO: 18 - protein, 7 aa, Artificial Sequence
  Synthetic
CEYSGER

SEQ ID NO: 19 - protein, 7 aa, Artificial Sequence
  Synthetic
CRKSSER

SEQ ID NO: 20 - protein, 7 aa, Artificial Sequence
  Synthetic
CEKSSER

SEQ ID NO: 21 - protein, 7 aa, Artificial Sequence
  Synthetic
CEKGSER

SEQ ID NO: 22 - protein, 7 aa, Artificial Sequence
  Synthetic
CRGSSER

SEQ ID NO: 23 - protein, 7 aa, Artificial Sequence
  Synthetic
CEFSSER

SEQ ID NO: 24 - protein, 7 aa, Artificial Sequence
  Synthetic
CEYSSER

SEQ ID NO: 25 - protein, 4 aa, Artificial Sequence
  Synthetic Gly-Ser linker
GGGS

SEQ ID NO: 26 - protein, 15 aa, Artificial Sequence
  Synthetic Gly-Ser linker
GGGGSGGGGSGGGGS

SEQ ID NO: 27 - protein, 5 aa, Artificial Sequence
  Synthetic Gly-Ser linker
GGGGS

SEQ ID NO: 28 - protein, 10 aa, Artificial Sequence
  Synthetic GlySer linker
GGGGSGGGGS

SEQ ID NO: 29 - protein, 20 aa, Artificial Sequence
  Synthetic Gly-Ser linker
GGGGSGGGGSGGGGSGGGGS

SEQ ID NO: 30 - protein, 25 aa, Artificial Sequence
  Synthetic Gly-Ser linker
GGGGSGGGGSGGGGSGGGGSGGGGS